(19) United States
(12) Patent Application Publication
(10) Pub. No.: US 2026/0250623 A1
(43) Pub. Date: Aug. 27, 2026

(54) Non-Destructive, Non-Invasive Method for Identifying and Estimating the Pluripotency of Cell Colonies from Image Data

(71) Applicant: Sartorius BioAnalytical Instruments, Inc., Bohemia, NY (US)

(72) Inventors: Andrea Chatrian, Bologna (IT); Jasmine Trigg, Biggleswade (GB); Daniel Porto, Bohemia, NY (US); Gillian Lovell, St. Albans (GB)

(21) Appl. No.: 19/065,673

(22) Filed: Feb. 27, 2025

(51) Int. Cl. C12M 1/36; C12M 1/34; G06N 20/20; G06T 7/00; G06V 20/69

(52) U.S. Cl. CPC C12M 41/48; C12M 41/32; C12M 41/36; G06N 20/20; G06T 7/0012; G06V 20/695; G06V 20/698; G06T 2207/30024

(57) ABSTRACT

The disclosure provides example methods determining, non-destructively and in a label-free manner, a level of pluripotency of cell colonies within a cell sample using phase contrast or other microscopic image data taken of the cell sample. A machine learning model is used to determine the locations and extents of nuclei within the image, and the location and extent of cell colonies within the image is also determined. For a given cell colony, a descriptive feature vector is determined based on those of the nuclei that are located within the cell colony, along with the portion of the image that depicts the cell colony (e.g., the shape and size of the portion and/or the brightness or other image information about the portion). The descriptive feature vector is then applied to another machine learning model to predict, for the cell sample, the pluripotency level.

### BACKGROUND

Pluripotent stem cells find beneficial use in many applications. However, inducing and maintaining pluripotency in cells is difficult, with the result that a cell sample cultured to include only pluripotent cells may include many (or even only) non-pluripotent cells. This can lead to reduced yields from downstream processes or other unwanted effects. For example, differentiation of pluripotent cells into a target differentiated cell type can be accomplished by applying a specified sequence of interventions to a pluripotent cell sample (e.g., applying a sequence of growth or differentiation factors, or other incubation conditions); however, non-pluripotent cells present at the beginning of such a sequence may fail to differentiate into the target cell type and/or may interfere with the successful differentiation of other cells in the sample, leading to reduced yields. It is possible to experimentally measure the degree of pluripotency of a cell sample via sequencing, the application and imaging of pluripotency-specific labels, or other interventions. However, such interventions introduce significant modifications to the sample, and may be destructive, limiting their utility (e.g., to inferring the degree of pluripotency of sibling, non-assessed samples that were exposed to similar incubation conditions).

### SUMMARY

In one aspect, an example label-free method for non-destructively detecting pluripotency of cell colonies within a cell sample is disclosed. The method includes: (i) obtaining a phase contrast image of a cell sample that contains stem cells; (ii) applying the image to a first machine learning model to determine a set of locations and extents of cell nuclei within the image; (iii) based on the phase contrast image, determining a first location and extent of a first cell colony of a plurality of cell colonies within the image; (iv) based on the portion of the image that is within the first location and extent of the first cell colony and those of the determined set of locations and extents of cell nuclei that are within the location and extent of the first cell colony, determining a first set of features for the first cell colony; and (v) applying the first set of features to a second machine learning model to predict a first level of pluripotency of the first cell colony.

In another aspect, an example non-transitory computer-readable medium is disclosed. The computer readable medium has stored thereon program instructions that upon execution by a processor, cause performance of one or more of the above methods.

In a still further aspect, a system is provided that includes: (i) at least one processor; and (ii) a non-transitory computer-readable medium, having stored therein instructions executable by the at least one processor to cause the system to perform one or more of the above methods. Such a system can include or be in communication with a phase contrast imager or other imaging system, e.g., to allow images to be obtained from one or more cell samples.

In yet another aspect, a system for non-destructively detecting pluripotency of a plurality of cell colonies within a live cell sample is provided. The system includes: (i) at least one processor; (ii) an incubator; (iii) a phase contrast imager; and (iv) a non-transitory computer-readable medium. The non-transitory computer-readable medium has stored therein instructions executable by the at least one processor to cause the system to perform a method comprising: (i) operating the phase contrast imager to obtain a phase contrast image of the live cell sample that contains stem cells and that is located in the incubator; (ii) applying the image to a first machine learning model to generate a mask indicative of the presence of cell nuclei within the image; (iii) determining, based on the mask, locations and extents of cell nuclei within the image; (iv) applying the image to a second machine learning model to determine locations and extents of the plurality of cell colonies within the image; (v) based on the portion of the image that is within the first location and extent of a first cell colony and those of the determined set of locations and extents of cell nuclei that are within the location and extent of the first cell colony; and (vi) determining a plurality of respective levels of pluripotency for the plurality of cell colonies, wherein determining a level of pluripotency of a particular cell colony of the plurality of cell colonies comprises: (a) determining a set of features for the particular cell colony based on the portion of the image that is within the determined location and extent of the particular cell colony and those of the determined locations and extents of cell nuclei that are within the location and extent of the particular cell colony, and (b) applying the set of features to a third machine learning model to predict a level of pluripotency of the particular cell colony.

The features, functions, and advantages that have been discussed can be achieved independently in various examples or may be combined in yet other examples further details of which can be seen with reference to the following description and drawings.

### BRIEF DESCRIPTION OF THE DRAWINGS

FIG. 1A depicts a block diagram of aspects of a method, according to an example implementation;

FIG. 1B depicts a phase contrast image of a cell sample and a prediction of the pluripotency of cell colonies thereof, according to an example implementation;

FIG. 2A depicts a fluorescence image of a cell sample and a prediction of the location of nuclei thereof, according to an example implementation;

FIG. 2B depicts a fluorescence image of a cell sample and a prediction of the location of nuclei thereof, according to an example implementation;

FIG. 3A depicts the segmentation of a cell sample into cell colonies, according to an example implementation;

FIG. 3B depicts the segmentation of a cell sample into cell colonies, according to an example implementation;

FIG. 3C depicts experimental results demonstrating loss of pluripotency when cells are cultured in non-optimised media for 2 days (left) and 4 days (right);

FIG. 3D depicts experimental results illustrating the thresholds chosen to classify the cells to generate the results of FIG. 3C;

FIG. 3E depicts experimental results;

FIG. 3F depicts experimental results;

FIG. 4A depicts predictions of the pluripotency of cell colonies of two different cell samples over time, according to an example implementation;

FIG. 4B depicts the ability of various predictor models to predict the pluripotency of cell colonies of cell samples, according to an example implementation;

FIG. 5 is a functional block diagram of a system, according to one example implementation;

FIG. 6 depicts a block diagram of a computing device and a computer network, according to an example implementation;

FIG. 7 shows a flowchart of a method, according to an example implementation.

The drawings are for the purpose of illustrating examples, but it is understood that the inventions are not limited to the arrangements and instrumentalities shown in the drawings.

### DETAILED DESCRIPTION

### I. Overview

It is desirable to generate pluripotent stem cells in order to facilitate a variety of scientific, therapeutic, or other applications. However, inducing and/or maintaining pluripotency in a sample of cells is difficult. A variety of preparations are known to induce and/or maintain pluripotency in cell samples of a variety of types. However, these techniques are not perfect, and can result in samples that include many, or even exclusively, non-pluripotent cells. It can be wasteful to use such non-pluripotent cell samples for downstream processes or analyses (e.g., a specified preparation to differentiate pluripotent cells into a specified target cell type or mixture of cell types), as the resources expended may result in reduced yields. Further, non-pluripotent cells in such a sample could have unwanted effects on the pluripotent cells.

It is possible to use labels and/or destructive processes to experimentally assess cell samples and/or colonies within cell samples with respect to their pluripotency. These assessments can then be used to determine which samples, or which portions of samples, to use for downstream processes (e.g., to differentiate into samples of target cell types, to use for scientific assessments). However, the use of labels to assess the pluripotency of cells (either by addition of fluorophores or other labels to the sample, or by genetically modifying the sample to express the label endogenously) can have significant perturbative effects on the cells, modifying their behavior and potentially causing them to lose pluripotency. Alternatively, the use of destructive testing (e.g., sequencing) results in the directly-assessed sample being destroyed, limiting the pluripotency predictions determined thereby to use inferentially on cell samples subject to similar incubation conditions which may not exhibit the same levels of pluripotency as the destructively assessed sample.

The methods described herein are label-free, non-destructive techniques that allow the level of pluripotency of samples of mesenchymal stem cells, embryonic stem cells, induced pluripotent stem cells, or other types of stem cells to be directly assessed via phase contrast imaging without significantly perturbing the samples. Indeed, such samples can be phase contrast imaged without being removed from the incubator (e.g., using in-incubator robotic, microscopic imaging apparatus), further reducing the level of perturbation associated with the pluripotency-assessment methods described herein. Such direct, non- or minimally-perturbative methods allow the pluripotency of specific cell colonies to be assessed and then used to inform interventions on the same cell colonies (e.g., modifying an incubation method to improve the induction or maintenance of pluripotency, using a cell picker to extract only pluripotent cells from a sample) and/or to repeatedly assess a cell sample over time (e.g., to generate a complete record of the pluripotency of the sample and/or cell colonies thereof over time for quality control or other purposes).

The methods described herein accomplish these benefits by using phase contrast imagery (or other microscopic image data) to identify both (i) the locations and extents of cell nuclei within a cell sample, and (ii) the locations and extents of cell colonies within the phase cell sample. This information is then used to determine a set of features (e.g., a vector of features) for each cell colony based on (i) those of the identified cell nuclei that are within each cell colony, and (ii) the portion of the phase contrast image that is within each cell colony (which can include features based on the geometry of the cell colony, e.g., an area, largest linear dimension, roundness, or other properties of the shape of the cell colony). The set of features for each cell colony are then applied to a machine learning model to predict, for each cell colony, a respective level of pluripotency.

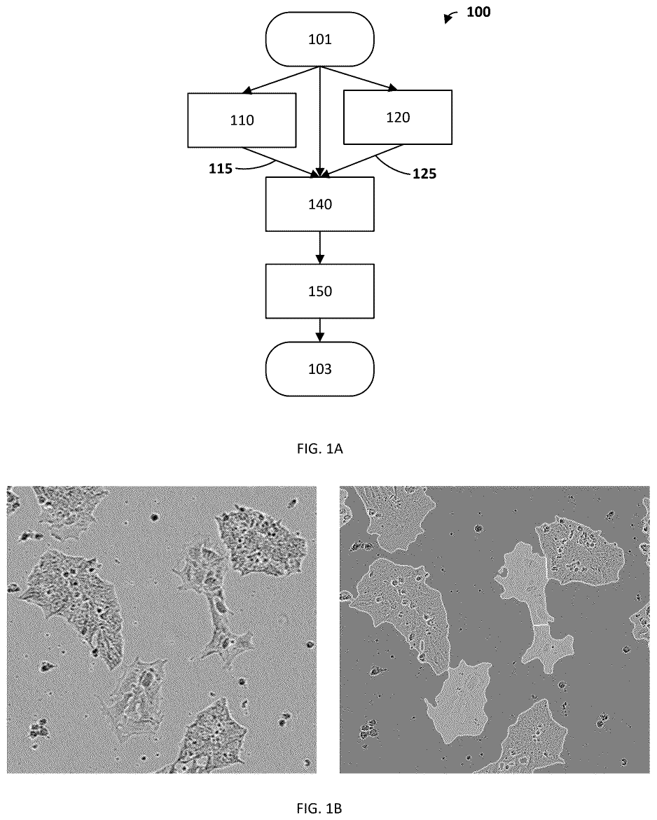

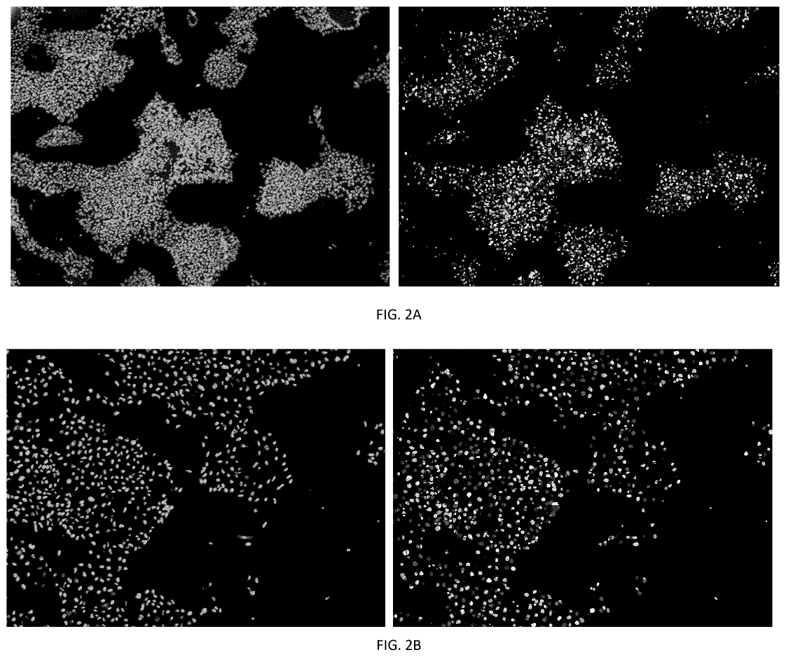

FIG. 1A depicts aspects of such a pluripotency level-determining process 100. An input image 101 (e.g., a phase contrast image) is applied to a first machine learning model 110 to determine a set of locations and extents of cell nuclei 115 within the image 101. The input image 101 is also used, by a colony detector 120, to determine the location(s) and extent(s) of one or more cell colonies 125, of a plurality of cell colonies, within the image 101. The determined set of locations and extents of cell nuclei 115, the input image 101, and the location(s) and extent(s) of the one or more cell colonies 125 are then used by a feature extractor 140 to determine one or more respective sets of features for the one or more cell colonies. Each set of features is then applied to a second machine learning model 150 to output respective predicted pluripotency levels 103 for each of the one or more cell colonies depicted in the input image 101.

FIG. 1B depicts an example input phase contrast image (left) and an indication of pluripotency levels determined therefrom for cell colonies depicted therein (right). The extents of the identified cell colonies are depicted by slightly lightened lines that encircle each cell colony (including one group of three colonies that are in contiguous with each other, and another pair of colonies that are in contact with each other). The pluripotency level (in this case, a binary label indicating either “pluripotent” or “not pluripotent”) for each colony is depicted by its coloration; lighter colonies (including the three central colonies within the image) are “not pluripotent,” while darker colonies are “pluripotent.” As depicted in FIG. 1B, the methods herein can be used to predict the pluripotency levels of cell colonies that are individual (i.e., not contacting other colonies) within a cell sample and of cell colonies that are in contact with (and, indeed, could be fully surrounded by) other cell colonies within a cell sample.

As noted above, a set of features is determined for each identified cell colony in a cell sample image. These features, for a given cell colony, could include a variety of features determined based on the set of locations and extents of cell nuclei within the image that are located within the given cell colony and/or based on the portion of the image that is within the determined location and extent of the given cell colony. Features based on the set of locations and extents of cell nuclei within the image that are located within the given cell colony could include a number of the cell nuclei, a total area of the cell nuclei, a pattern of distribution of the cell nuclei (e.g., an average inter-nucleus distance, a minimum inter-nucleus distance), or an average, range, standard deviation, or other property of a distribution across the nuclei with respect to area, roundness, eccentricity, solidity, or some other per-nucleus property. Features based on the portion of the image that is within the determined location and extent of the given cell colony could include features relating to the geometry/shape of the portion of the image that is within the determined location and extent (e.g., an area, a convex area, an eccentricity, an extent or other measure of a linear dimension of the given cell colony) and/or features relating to the pixels or other contents of the sample image within the determined location and extent of the given cell colony (e.g., a mean, minimum, maximum, or percentile value of an intensity of the portion of the image that is within the location and extent of the given cell colony, a solidity of the given cell colony, a Hu moment of the portion of the image that is within the location and extent of the given cell colony, a weighted Hu moment of the portion of the image that is within the location and extent of the given cell colony, a Haralick texture metric of the portion of the image that is within the location and extent of the given cell colony). Features based on the set of locations and extents of cell nuclei within the image that are located within the given cell colony and the portion of the image that is within the determined location and extent of the given cell colony could include a nucleus-to-cytoplasm ratio of the given cell colony.

The set of features determined for the given cell colony could include all or a subset of the above features, and/or could include additional or alternative features to those listed above. The set of features determined for the given cell colony could include all of, at least ten of, at least five of, or at least one of: an area of the given cell colony, a convex area of the given cell colony, an eccentricity of the given cell colony, an extent of the given cell colony, a mean, minimum, maximum, or percentile value of an intensity of the portion of the image that is within the location and extent of the given cell colony, a perimeter length of the given cell colony, a solidity of the given cell colony, a Hu moment of the portion of the image that is within the location and extent of the given cell colony, a weighted Hu moment of the portion of the image that is within the location and extent of the given cell colony, a Haralick texture metric of the portion of the image that is within the location and extent of the given cell colony, a number of the nuclei within the given cell colony, a mean area of the nuclei within the given cell colony, a standard deviation of the areas of the nuclei within the given cell colony, a solidity of the nuclei within the given cell colony, or a nucleus-to-cytoplasm ratio of the given cell colony.

The set of locations and extents of nuclei 115 within the input image 101 could be determined therefrom, using the first machine learning model 110, in a variety of ways. Additionally, the first machine learning model 110 could have a variety of architectures and could be trained in a variety of ways based on a variety of types of training data. The first machine learning model 110 could be trained to output the set of locations and extents 115 directly (e.g., as a segmentation map, as a set of X and Y coordinate locations and additional information indicative of the geometry and size of each of the nuclei) or indirectly. For example, the first machine learning model 110 could be trained on pairs of images, with each pair of images including (i) a phase-contrast image of a respective cell sample, and (ii) an image of a nucleus-specific fluorophore or dye within the respective cell sample. The first machine learning model 110 could then be trained to generate, as an output, a predicted pattern image of the nucleus-specific fluorophore or dye based on an input phase contrast image. Such an output of the trained first machine learning model 110 could then be used to determine the set of locations and extents of cell nuclei 115 within the input image 101 by applying threshold, inpainting, greedy algorithms, or other segmentation methods to generate a segmentation map of nuclei within the input image 101 and/or to generate some other representation of the locations and extents of cell nuclei 115 within the input image 101.

FIG. 2A depicts an experimental result of such training, with the left image being a ‘ground truth’ fluorescence image of nucleus-specific dye within a cell sample containing induced pluripotent stem cells, and the right image being a prediction of the locations and extents of cell nuclei within the cell sample. FIG. 2B depicts another experimental result of such training, with the left image being a ‘ground truth’ fluorescence image of nucleus-specific dye within a cell sample containing induced pluripotent stem cells that have largely become non-pluripotent, and the right image being a prediction of the locations and extents of cell nuclei within the cell sample.

The location(s) and extent(s) of one or more cell colonies 125 in the input image 101 could be determined therefrom in a variety of ways. For example, the colony detector 120 could include a machine learning model or other method (e.g., thresholding followed by inpainting/filtering) could be used to determine the location(s) and extent(s) of contiguous cell mass(es) within the image. The location(s) and extent(s) of one or more cell colonies 125 within each such contiguous cell mass could then be determined by segmenting the contiguous cell mass into multiple non-overlapping sub-regions. For example, a watershed algorithm could be applied to each contiguous cell mass to determine therefrom the location(s) and extent(s) of one or more sub-regions (and thus, cell colonies) composing each contiguous cell mass.

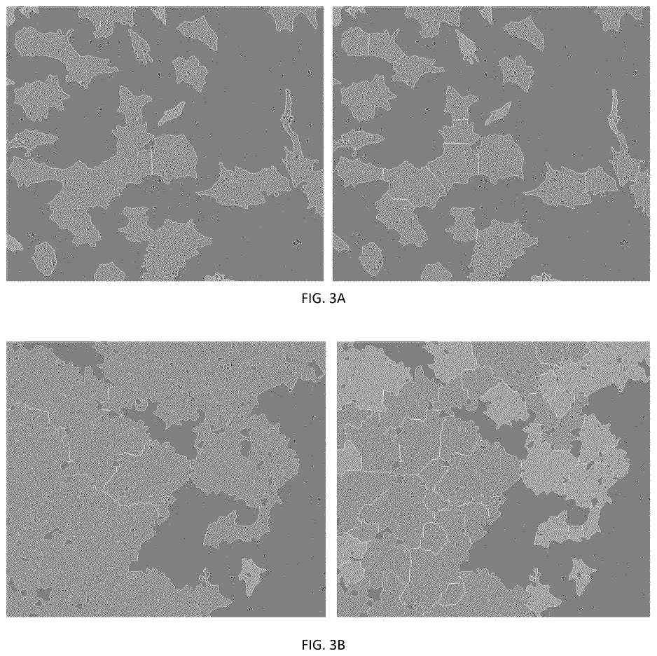

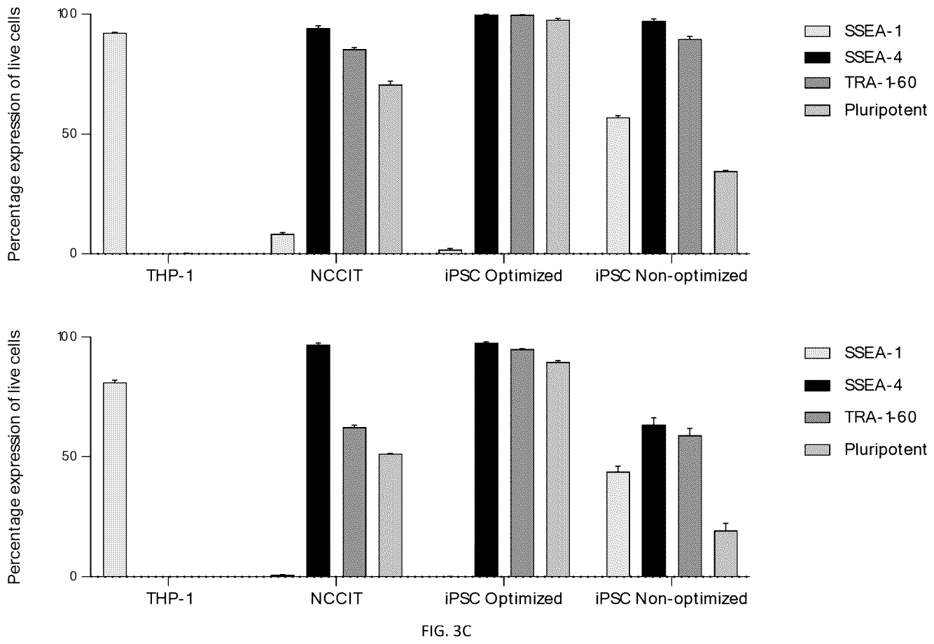

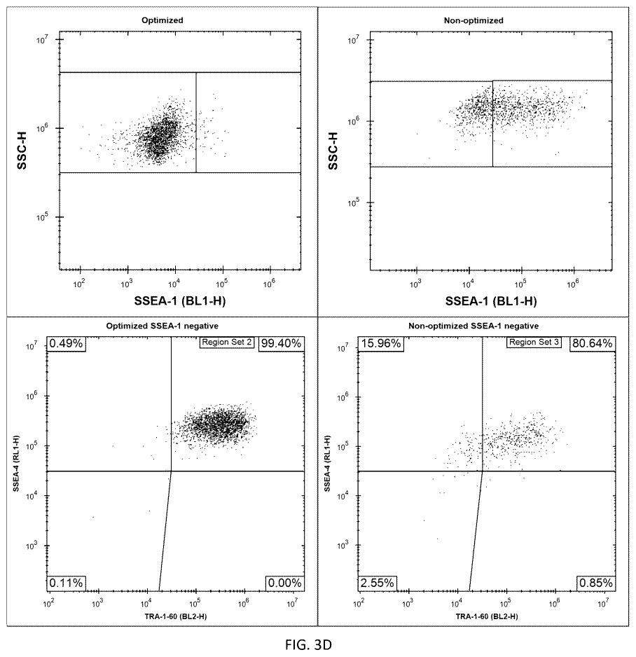

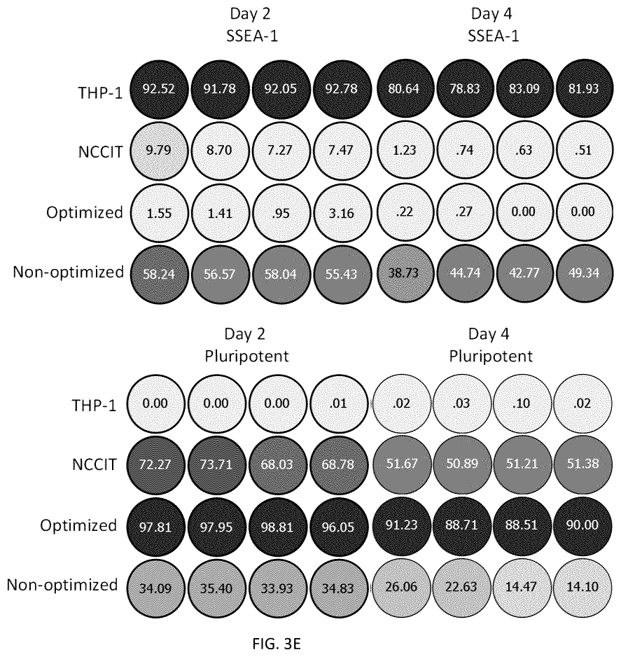

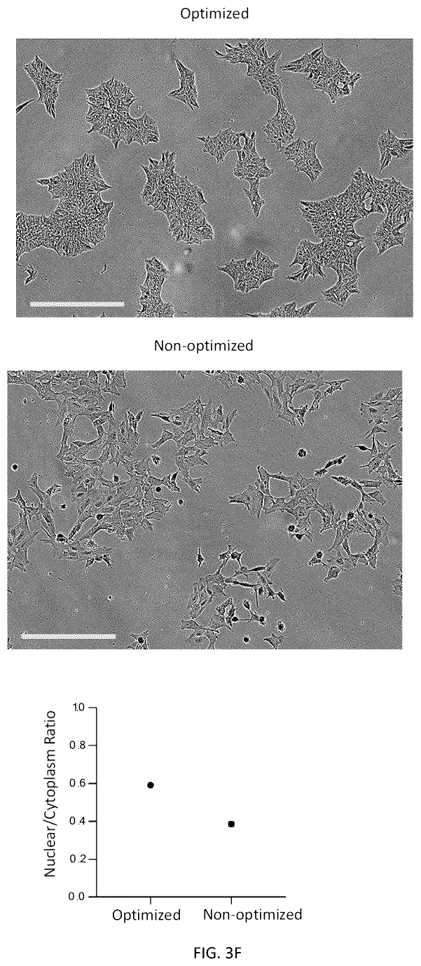

FIG. 3A depicts an experimental result of the use of such a watershed algorithm on an input phase contrast image, with the left image being the segmentation of a cell sample containing induced pluripotent stem cells into cell colonies according to a first value of a watershed partition parameter, and the right image being the segmentation of the cell sample into cell colonies according to a second, smaller value of the watershed partition parameter. FIG. 3B depicts an experimental result of the use of such a watershed algorithm on an input phase contrast image, with the left image being the segmentation of a cell sample containing induced pluripotent stem cells that have largely become non-pluripotent into cell colonies according to the first value of a watershed partition parameter, and the right image being the segmentation of the cell sample into cell colonies according to the second, smaller value of the watershed partition parameter.

Once the set of features have been determined for a particular cell colony within the image 101 have been generated (e.g., by performing appropriate computations based on the location and extent of the particular cell colony, the locations and extents of cell nuclei located within the location and extent of the particular cell colony, and/or the portion of the image 101 within the location and extent of the particular cell colony), they can be applied to the second machine learning model 150 to generate, for the particular cell colony, a level of pluripotency 103. The level of pluripotency 103 can be a categorical value (e.g., binary, representing “pluripotent” or “not pluripotent,” or more categories, e.g., “most pluripotent,” “marginally pluripotent,” or “not pluripotent”) or a variable value (e.g., a continuous or discrete-values numerical value that represents, within a specified range (e.g., 0 to 1), the degree of pluripotency of the particular cell colony).

The second machine learning model 150 can be configured in a variety of ways (e.g., a variety of different architectures) and trained in a variety of different ways, based on a variety of different types of training data, to be able to predict, from an input set of features, a pluripotency level for the cell colony whose set of features was input. For example, the second machine learning model could include an artificial neural network, a support vector machine, a clustering method (e.g., k-means), one or more linear or nonlinear mappings (e.g., a nonlinear kernel used to map the input features into a different space having the same, more, or fewer dimensions than the number of features), a decision tree, a regression tree, a Bayesian classifier, or some other aspect or element of a machine learning model or combination of such. In some examples, the second machine learning model could include a plurality of sub-models whose outputs are all combined (e.g., by averaging, by voting) to result in a single output; such an aggregate of sub-models whose outputs are combined in some manner to generate an overall model output can be referred to as an ensemble model. Such an ensemble model could include a regression forest (i.e., an aggregate of regression trees), a random forest (i.e., an aggregate of decision trees and/or regression trees), an ensemble of Bayesian classifiers, or some other collection of sub-models whose respective outputs are used to generate an overall output for the ensemble model (e.g., by averaging, weighted averaging, summing, determination of a median, determination of one or more moments of a distribution of the outputs, voting or otherwise selecting a most commonly-generated output). Such ensemble models could be trained and/or combined in a variety of ways, e.g., via boosting, bagging, or other methods.

The second machine learning model 150 could be trained based on a variety of training data. For example, the second machine learning model could be trained on sets of phase contrast (or other microscopic imagery) of cell samples and corresponding ‘ground truth’ data for the cell colonies within the cell samples. Such ground truth data could be determined at the whole-sample level (e.g., sequencing, flow cytometry (e.g., to detect markers of pluripotency like SSEA-4 and/or TRA-1-60), or some other experimental assessment of the whole sample to generate an overall pluripotency level for all of the colonies in common) or at the individual cell colony level (e.g., spatially-selective sequencing to determine a level of pluripotency that varies across the sample, and thus can apply different pluripotency levels to different cell colonies according to their location, or via imaging live-cell compatible fluorescently labelled antibodies that bind to surface markers indicative of pluripotency, e.g., SSEA-4 or TRA-1-60). In some examples, the pluripotency level for a whole sample could be inferred, e.g., based on an experimental treatment applied to the whole sample such that it is likely (or unlikely) at the timing of the phase contrast imaging of the sample that the sample, as a whole, has a high (or low) pluripotency level. For example, a phase contrast image taken after 48 hours of incubation in mTESR, a culture medium designed to maintain pluripotency in an induced pluripotent stem cell sample, could be labeled with a ground truth “high pluripotency level,” while a phase contrast image taken after 48 hours of incubation when RPMI has been applied to an induced pluripotent stem cell sample to induce loss of pluripotency could be labeled with a ground truth “low pluripotency level.”

FIGS. 3C-F depict experimental results relating to culturing the cell samples in mTESR/RPMI medium as a proxy for pluripotency of iPSC cell samples, and the relationship between expression of SSEA-1, SSEA-4, and TRA-1-60 and pluripotency. FIG. 3C depicts the pattern of expression in live cells of three different markers (a “normal” marker SSEA-1 and two pluripotency markers SSEA-4, and TRA-1-60) across four different culture conditions (two control cell lines, THP-1 and NCCIT in generic culture medium, and an iPSC cell line with pluripotency induced therein and cultured in an “Optimized” mTESR medium and a “Non-optimized” RPMI medium) and at two different points in time (two and four days). FIG. 3D depicts flow cytometry sample data and the gating strategy used to generate the data for FIG. 3C. As illustrated in FIGS. 3C-D, an increase in “normal” marker (SSEA-1) expression was observed when cells were grown in RPMI, along with decreases in expression of SSEA-4 and TRA-1-60, indicating a loss of pluripotency. This corresponds to changes in the ‘Pluripotent’ bars of FIG. 3C (positive for both SSEA-4 and TRA-1-60). FIG. 3E depicts a ‘heatmap’ of the sample wells used to generate the data for FIGS. 3C and 3D, illustrating the percentage of cells expressing SSEA-1 or the ‘Pluripotent’ phenotype in each well of the 96-well plate used to generate the experimental data. FIG. 3F depicts phase contrast microscopy images of the morphology of iPSCs grown in mTESR (“Optimized”) or RPMI (“Non-optimized”) for 2 days and the nuclear/cytoplasm ratio associated with those growth conditions.

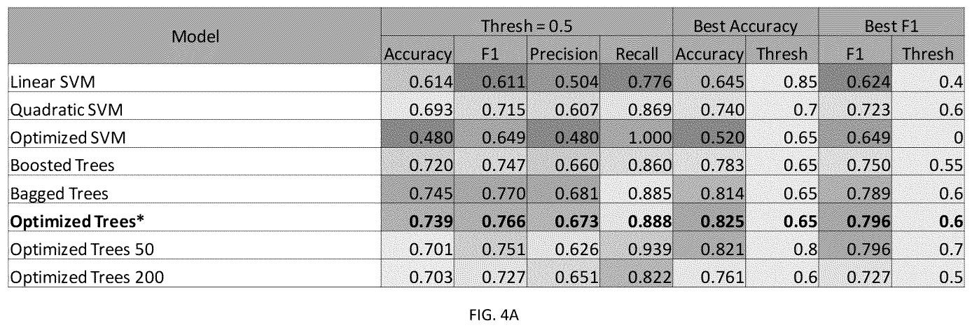

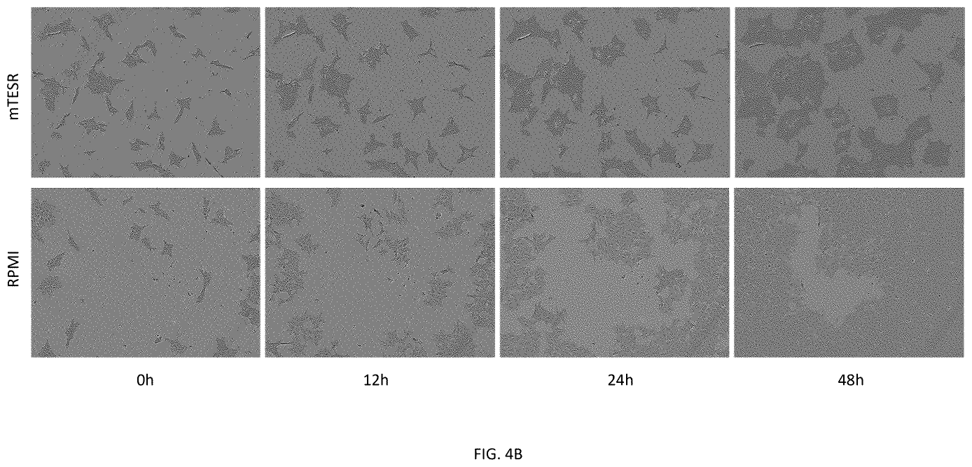

FIG. 4A depicts experimentally evaluated accuracy and other outcome measures for a variety of different types of machine learning models used, as described herein, to predict pluripotency levels for cell colonies based on phase contrast images thereof.

Pluripotency levels determined for each cell colony of a cell sample as described herein could be used to facilitate a variety of applications, e.g., related to the ability of the methods described herein to generate such levels in a label-free, non-destructive manner. Thus, such assessments can be performed multiple times for the same sample (e.g., one or more times per day, or according to some other schedule). FIG. 4B illustrates the use of these methods to image the same samples (on top, a sample of induced pluripotent stem cells incubated in mTESR to maintain pluripotency, and on the bottom, a sample of induced pluripotent stem cells cultured in media with RPMI added thereto to induce spontaneous loss of pluripotency) at multiple time points (a first image at time ‘0’, then at 12, 24, and 48 hours later). Lighter-colored cell colonies have been determined, via the methods described herein, to have ‘low’ pluripotency levels, while darker-colored cell colonies have been determined, via the methods described herein, to have ‘high’ pluripotency levels. Thus, at the first time point, almost all of the colonies in both samples have ‘high’ levels, while at the 48 hour mark, culturing in mTESR has allowed almost all of the colonies to retain that high level, while all of the colonies in the sample exposed to RPMI have assumed a low pluripotency level.

Such high temporal resolution measures of pluripotency level can be used for quality assurance of cell samples or for some other application as part of a record made for the sample (e.g., for each cell colony in the sample, along with information indicating the location or other defining information for each of the colonies in the sample). This could be done to provide assurances to a recipient of the samples and/or to select which of a set of samples will be used (e.g., provided to recipient(s)) and which will be discarded (e.g., due to having sufficient numbers of low pluripotency level cell colonies). Such data can also be used to facilitate scientific or other assessments of the cell samples. For example, such high temporal resolution data could be used to enhance downstream treatments/analyses by allowing those treatments/analysis to be tailored to the specific patterns of pluripotency level exhibited over time (e.g., applying a first cell differentiation protocol to samples that exhibited consistently high pluripotency levels across the assessment period, and applying a second, modified differentiation protocol to samples that exhibited lower or otherwise variable pluripotency levels across the assessment period).

Such high temporal resolution measures of pluripotency level can be used to indicate, in realtime or near realtime, change in and/or loss of pluripotency across one or more wells of a multi-well sample plate and/or across one or more colonies identified within a cell sample (e.g., within a well of a multi-well sample plate). For example, an indication of a loss or other change in pluripotency of a single colony of a cell sample, or of an overall level of pluripotency of a cell sample (e.g., an average of pluripotency values determined for colonies within the sample) could be provided as soon as such a change is predicted, e.g., within seconds or milliseconds of the acquisition and subsequent processing of a phase contrast image of the sample as described herein. Such low-latency feedback on the pluripotency state of cell samples currently undergoing incubation can allow measures to be taken swiftly to preserve, or even regain, the pluripotency of cell samples by adjusting one or more parameters of the incubation (e.g., temperature light level, pressure, humidity), adding, subtracting, or adjusting an ongoing rate of provision of one or more substances (e.g., cell culture medium, sugars, water, buffer), or taking some other action that, as a result of the low-latency estimate of pluripotency levels, is able to increase the overall pluripotency level of one or more samples.

Such high temporal resolution data could be used to improve methods of inducing and/or maintaining the pluripotency of cell samples (e.g., by modifying a standard protocol for such). This increased data on pluripotency over time can allow more subtle distinctions between different treatments to be detected that might not have been detectable based only on an endpoint assessment of pluripotency. Additionally or alternatively, the pluripotency levels determined for a cell sample could be used to provide feedback on an ongoing incubation in order to, e.g., increase the percentage of the cell colonies that exhibit a high level of pluripotency at the end of the incubation or that are improved in some other way. For example, a substance (e.g., growth medium, buffer, cell signaling compound(s)) could be added to a cell sample, or a rate of ongoing provision of a substance (e.g., oxygen, carbon dioxide, water, sugars, growth medium, cell signaling compound(s)) could be increased or decreased, based on one or more pluripotency levels determined using the methods described herein. Using such feedback to tailor the ongoing incubation parameters of a cell sample can result in increased yields, increased overall levels of pluripotency, or other improvements to samples of induced pluripotent stem cells, mesenchymal stem cells, or embryonic stem cells, or other types of stem cells generated using the methods described herein.

Cell colony-specific pluripotency levels determined as described herein can also be used to identify specific cell colonies from a cell sample (e.g., cell colonies that exhibit high pluripotency levels) for use in downstream processes. For example, a robotic cell picker or other apparatus could be used to extract cells from selected cell colonies (e.g., from colonies having high pluripotency values) and not from non-selected cell colonies (e.g., not from colonies having low pluripotency values) in order to increase yield or otherwise improve downstream processes by ensuring that only highly pluripotent cells are used therefor.

### II. Example Architecture

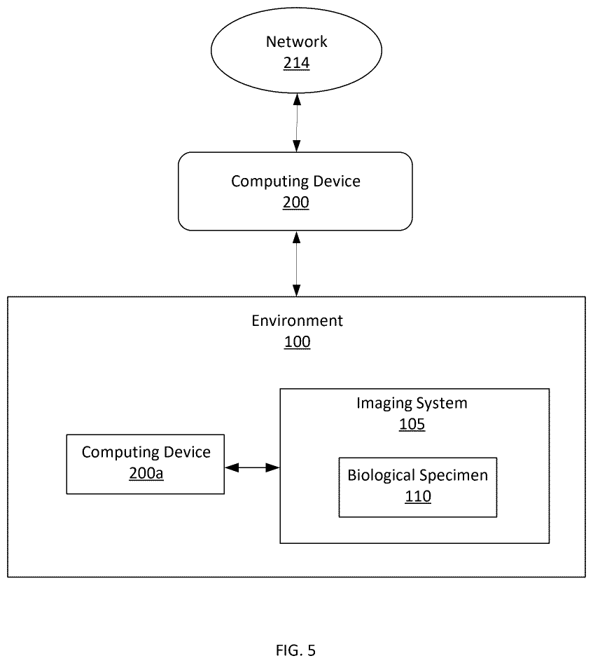

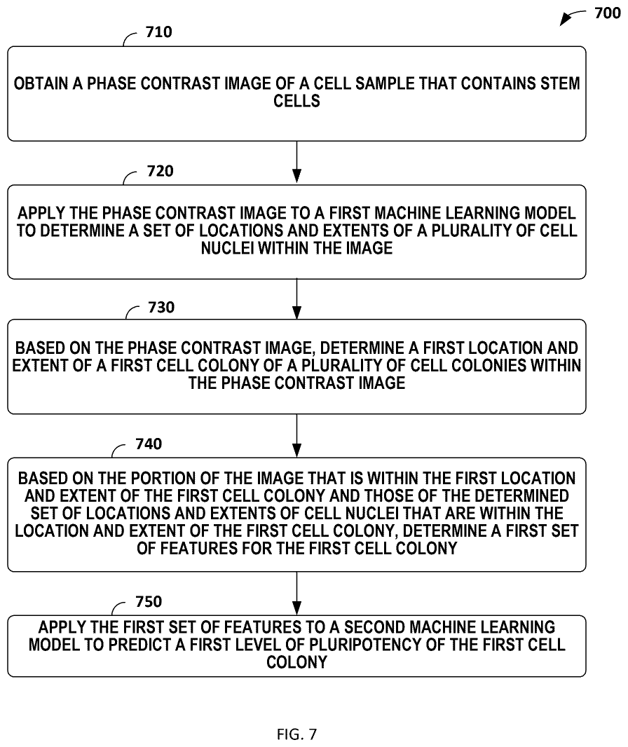

FIG. 5 is a block diagram showing an operating environment 100 that includes or involves, for example, a phase contrast imager or other imaging system 105 and a biological specimen 110 having a plurality of cells. Method 700 in FIG. 7 described below shows embodiments of method that can be implemented within this operating environment 100.

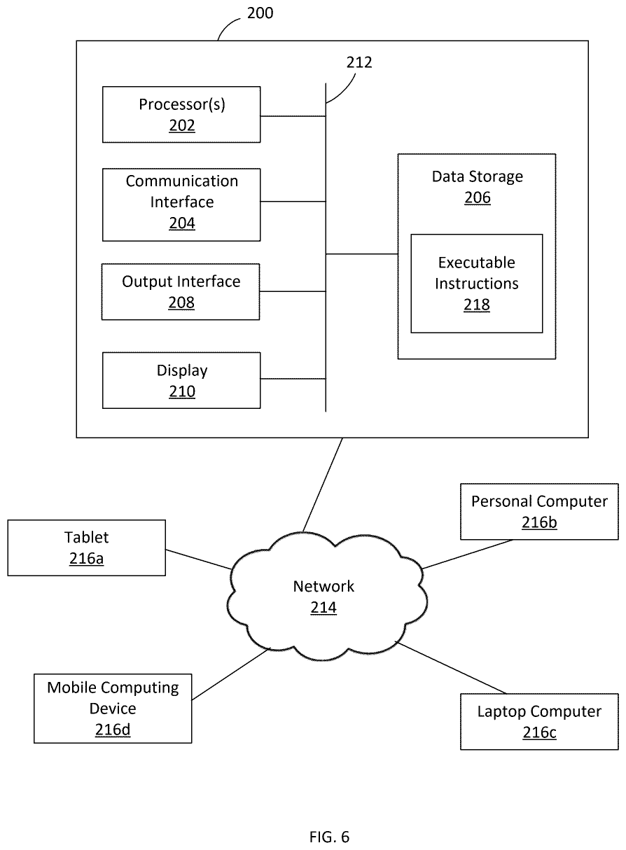

FIG. 6 is a block diagram illustrating an example of a computing device 200, according to an example implementation, that is configured to interface with operating environment 100, either directly or indirectly. The computing device 200 may be used to perform functions of methods described herein, e.g., those shown in FIG. 7 and described below. Computing device 200 can be configured to perform one or more functions, including image processing, executing machine learning models (e.g., to process input images), applying watershed or other applications to segment a region (e.g., a region of cells into multiple different cell colonies), determining sets of features for cell colonies in an image (e.g., based on the geometry of the cell colonies, image contents of the cell colonies, the number and other properties of cell nuclei detected within the cell colonies) the application of feature sets to a machine learning model to predict a level of pluripotency of a cell colony, classification, model training, display, or other functions that are based, in part, on phase contrast or other images of cells obtained by the imaging system 105, for example. The computing device 200 has a processor(s) 202, and also a communication interface 204, data storage 206, an output interface 208, and a display 210 each connected to a communication bus 212. The computing device 200 may also include hardware to enable communication within the computing device 200 and between the computing device 200 and other devices (e.g. not shown). The hardware may include transmitters, receivers, and antennas, for example.

The communication interface 204 may be a wireless interface and/or one or more wired interfaces that allow for both short-range communication and long-range communication to one or more networks 214 or to one or more remote computing devices 216 (e.g., a tablet 216a, a personal computer 216b, a laptop computer 216c and a mobile computing device 216d, for example). Such wireless interfaces may provide for communication under one or more wireless communication protocols, such as Bluetooth, Wi-Fi (e.g., an institute of electrical and electronic engineers (IEEE) 802.11 protocol), Long-Term Evolution (LTE), cellular communications, near-field communication (NFC), and/or other wireless communication protocols. Such wired interfaces may include Ethernet interface, a Universal Serial Bus (USB) interface, or similar interface to communicate via a wire, a twisted pair of wires, a coaxial cable, an optical link, a fiber-optic link, or other physical connection to a wired network. Thus, the communication interface 204 may be configured to receive input data from one or more devices and may also be configured to send output data to other devices.

The communication interface 204 may also include a user-input device, such as a keyboard, a keypad, a touch screen, a touch pad, a computer mouse, a track ball and/or other similar devices, for example.

The data storage 206 may include or take the form of one or more computer-readable storage media that can be read or accessed by the processor(s) 202. The computer-readable storage media can include volatile and/or non-volatile storage components, such as optical, magnetic, organic or other memory or disc storage, which can be integrated in whole or in part with the processor(s) 202. The data storage 206 is considered non-transitory computer readable media. In some examples, the data storage 206 can be implemented using a single physical device (e.g., one optical, magnetic, organic or other memory or disc storage unit), while in other examples, the data storage 206 can be implemented using two or more physical devices.

The data storage 206 is a non-transitory computer readable storage medium, and executable instructions 218 are stored thereon. The executable instructions 218 include computer executable code. When the instructions 218 are executed by the processor(s) 202, the processor(s) 202 are caused to perform functions. Such functions include, but are not limited to, operating the imaging system 105 to obtain phase contrast or other images of the biological specimen 110 (e.g., while the biological specimen 110 is located within an incubator). Such functions could additionally or alternatively include functions to apply such imaging data to one or more machine learning models, and/or perform some other computational tasks, to identify cell colonies within the biological specimen 110 and to predict for one or more of the identified cell colonies (e.g., for all of the cell colonies) a respective estimate of the level of pluripotency of the cells in each cell colony (e.g., a categorical label indicative of pluripotency level, a discrete or continuous-valued output indicative of pluripotency level), and/or to perform some other computation as described herein.

The processor(s) 202 may be a general-purpose processor or a special purpose processor (e.g., digital signal processors, application specific integrated circuits, etc.). The processor(s) 202 may receive inputs from the communication interface 204 and process the inputs to generate outputs that are stored in the data storage 206 and output to the display 210. The processor(s) 202 can be configured to execute the executable instructions 218 (e.g., computer-readable program instructions) that are stored in the data storage 206 and are executable to provide the functionality of the computing device 200 described herein.

The output interface 208 outputs information to the display 210 or to other components as well. Thus, the output interface 208 may be similar to the communication interface 204 and can be a wireless interface (e.g., transmitter) or a wired interface as well. The output interface 208 may send commands to one or more controllable devices, for example.

The computing device 200 shown in FIGS. 5 and 6 may also be representative of a local computing device 200a in operating environment 100, for example, in communication with imaging system 105. This local computing device 200a may perform one or more of the steps of the method 700 described below, may receive input from a user and/or may send cell count and/or optical property data and user input to computing device 200 to perform all or some of the steps of method 700.

FIG. 7 shows a flowchart of an example method 700 to determine, for one or more cell samples, respective estimated levels of pluripotency of cell colonies within the cell samples, according to an example implementation. Method 700 shown in FIG. 7 presents an exemplary method that can be used with the computing device 200 of FIG. 6, for example. Additionally or alternatively, some or all of the functionality of the methods described herein (e.g., method 700) could be performed by the servers, processors, or other elements of a cloud computing service, remote server, or other computing system that is remote from but in communication with the computing device 200 and/or imaging system 105 (and optionally additional such computing devices and/or imaging systems) via the network 214 (e.g., via the Internet). This could be beneficial in that such a remote computing system could have more extensive storage, database systems, memory, processing resources, or other computing resources to facilitate the training and/or inference of classifiers or other machine learning systems to operate on such cell sample image data, or performing some other computation or process as described herein. Such a remote (e.g., cloud-based) computing system could also have the benefit of access to many imaging systems or other sources of sample image data, allowing the remote system to train improved classifiers or other machine learning models or to otherwise improve the performance of the methods described herein.

Further, devices or systems may be used or configured to perform logical functions presented in FIG. 7. In some instances, components of the devices and/or systems may be configured to perform the functions such that the components are configured and structured with hardware and/or software to enable such performance. Components of the devices and/or systems may be arranged to be adapted to, capable of, or suited for performing the functions, such as when operated in a specific manner. Method 700 may include one or more operations, functions, or actions as illustrated by one or more of the blocks thereof. Although the blocks are illustrated in a sequential order, some of these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of the present examples. In this regard, each block may represent a module, a segment, or a portion of program code, which includes one or more instructions executable by a processor for implementing specific logical functions or steps in the process. The program code may be stored on any type of computer readable medium or data storage, for example, a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time such as register memory, processor cache and Random Access Memory (RAM). The computer readable medium may also include non-transitory media, such as secondary or persistent long-term storage, like read only memory (ROM), optical or magnetic disks, compact disc read only memory (CD-ROM), for example. The computer readable media may also be any other volatile or non-volatile storage systems. The computer readable medium may be considered a tangible computer readable storage medium, for example.

In addition, each block in FIG. 7, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

### III. Example Methods

Referring now to FIG. 7, a label-free method for non-destructively detecting pluripotency of cell colonies within a cell sample 700 is illustrated, optionally using the computing device of FIGS. 5-6. Method 700 includes, at block 710, obtaining a phase contrast image of a cell sample, such as a live cell sample, that contains stem cells. Then, at block 720, the method 700 includes applying the phase contrast image to a first machine learning model to determine a set of locations and extents of a plurality of cell nuclei within the image. Next, at block 730, the method 700 additionally includes, based on the phase contrast image, determining a first location and extent of a first cell colony of a plurality of cell colonies within the phase contrast image. The method 700 then includes, at block 740, based on the portion of the phase contrast image that is within the first location and extent of the first cell colony and those of the determined set of locations and extents of cell nuclei that are within the location and extent of the first cell colony, determining a first set of features for the first cell colony. The method 700 additionally includes, at block 750, applying the first set of features to a second machine learning model to predict a first level of pluripotency of the first cell colony.

The stem cells of the cell sample could include at least one of induced pluripotent stem cells, mesenchymal stem cells, or embryonic stem cells.

The second machine learning model could be or include an ensemble model.

Applying the phase contrast image to the first machine learning model to determine the set of locations and extents of cell nuclei within the image 720 can include: (i) applying the image to the first machine learning model to generate a predicted pattern of a nucleus-specific fluorophore within the cell sample; and (ii) segmenting the predicted pattern to determine the set of locations and extents of cell nuclei within the image.

Determining the first location and extent of the first cell colony within the phase contrast image 730 can include applying the phase contrast image to a third machine learning model to determine the first location and extent of the first cell colony within the image. Applying the phase contrast image to the third machine learning model to determine the first location and extent of the first cell colony within the image can include: (i) applying the phase contrast image to the third machine learning model to determine the location and extent of a contiguous cell mass within the image; and (ii) identifying respective locations and extents of a plurality of sub-regions of the determined location and extent of the contiguous cell mass, wherein the first location and extent of the first cell colony is the determined location and extent of one of the sub-regions of the contiguous cell mass.

In such examples, a determined second location and extent of a second cell colony of the plurality of cell colonies within the image is the determined location and extent of another one of the sub-regions of the contiguous cell mass, and the method 700 further includes: (i) based on the portion of the image that is within the second location and extent of the second cell colony and those of the determined set of locations and extents of cell nuclei that are within the second location and extent of the second cell colony, determining a second set of features for the second colony; and (ii) applying the second set of features to the second machine learning model to predict a second level of pluripotency of the second cell colony. In such examples, the method 700 can further include providing, on a display, an indication of the phase contrast image, the locations and extents of the first and second cell colonies within the phase contrast image, and the first and second levels of pluripotency of the first and second cell colonies, respectively. Additionally or alternatively, the method 700 can further include, responsive to determining that the first level of pluripotency exceeds a threshold value and that the second level of pluripotency does not exceed the threshold value, operating a robotic cell picker to extract cells from the first cell colony.

In some examples, the first set of features can include at least one of: an area of the first cell colony, a convex area of the first cell colony, an eccentricity of the first cell colony, an extent of the first cell colony, a mean, minimum, maximum, or percentile value of an intensity of the portion of the image that is within the first location and extent of the first cell colony, a perimeter length of the first cell colony, a solidity of the first cell colony, a Hu moment of the portion of the image that is within the first location and extent of the first cell colony, a weighted Hu moment of the portion of the image that is within the first location and extent of the first cell colony, a Haralick texture metric of the portion of the image that is within the first location and extent of the first cell colony, a number of the nuclei within the first cell colony, a mean area of the nuclei within the first cell colony, a standard deviation of the areas of the nuclei within the first cell colony, a solidity of the nuclei within the first cell colony, or a nucleus-to-cytoplasm ratio of the first cell colony.

In some examples, obtaining the phase contrast image of the cell sample 710 includes phase contrast imaging a live cell sample while the live cell sample is located within an incubator. In such examples, the phase contrast image is a first phase contrast image taken at a first point in time, the first level of pluripotency is a level of pluripotency of the first cell colony at the first point in time, and the method 700 further includes: (i) phase contrast imaging the cell sample at a second point in time while the cell sample is located within the incubator to obtain a second phase contrast image of the cell sample, wherein the second point in time is after the first point in time; (ii) applying the second image to the first machine learning model to determine a second set of locations and extents of cell nuclei within the second image; (iii) based on the second phase contrast image, determining a third location and extent of the first cell colony within the second image; (iii) based on the portion of the second image that is within the first location and extent of the first cell colony and those of the determined second set of locations and extents of cell nuclei that are within the third location and extent of the first cell colony, determining a third set of features for the first cell colony; and (iv) applying the third set of features to the second machine learning model to predict a third level of pluripotency of the first cell colony at the second point in time. Additionally or alternatively, the method 700 can further include, based on the first level of pluripotency and prior to the second point in time, at least one of (i) adjusting a rate at which a substance is continuously provided to the cell sample or (ii) providing a substance to the cell sample. Additionally or alternatively, the method 700 can further include, responsive to determining that the first level of pluripotency exceeds a threshold value, operating a robotic cell picker to extract cells from the first cell colony.

In some examples, the method 700 further includes: (i) while the cell sample is located within an incubator, obtaining an additional plurality of phase contrast images of the cell sample at a plurality of respective different points in time; (ii) based on the additional plurality of phase contrast images, using the first machine learning model and the second machine learning model to determine, for the first cell colony, a plurality of respective levels of pluripotency of the first cell colony at the respective different points in time; and (iii) storing the first level of pluripotency and the plurality of respective levels of pluripotency of the first colony in a quality control record for the cell sample.

The method 700 could include additional or alternative steps or features.

As discussed above, a non-transitory computer-readable medium having stored thereon program instructions that upon execution by a processor (e.g., 202) may be utilized to cause performance of any of the functions of the foregoing methods.

### IV. Conclusion

The above detailed description describes various features and functions of the disclosed systems, devices, and methods with reference to the accompanying figures. In the figures, similar symbols typically identify similar components, unless the context indicates otherwise. The illustrative embodiments described in the detailed description, figures, and claims are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the message flow diagrams, scenarios, and flowcharts in the figures and as discussed herein, each step, block and/or communication may represent a processing of information and/or a transmission of information in accordance with example embodiments. Alternative embodiments are included within the scope of these example embodiments. In these alternative embodiments, for example, functions described as steps, blocks, transmissions, communications, requests, responses, and/or messages may be executed out of order from that shown or discussed, including in substantially concurrent or in reverse order, depending on the functionality involved. Further, more or fewer steps, blocks and/or functions may be used with any of the message flow diagrams, scenarios, and flow charts discussed herein, and these message flow diagrams, scenarios, and flow charts may be combined with one another, in part or in whole.

A step or block that represents a processing of information may correspond to circuitry that can be configured to perform the specific logical functions of a herein-described method or technique. Alternatively or additionally, a step or block that represents a processing of information may correspond to a module, a segment, or a portion of program code (including related data). The program code may include one or more instructions executable by a processor for implementing specific logical functions or actions in the method or technique. The program code and/or related data may be stored on any type of computer-readable medium, such as a storage device, including a disk drive, a hard drive, or other storage media.

The computer-readable medium may also include non-transitory computer-readable media such as computer-readable media that stores data for short periods of time like register memory, processor cache, and/or random-access memory (RAM). The computer-readable media may also include non-transitory computer-readable media that stores program code and/or data for longer periods of time, such as secondary or persistent long-term storage, like read only memory (ROM), optical or magnetic disks, and/or compact-disc read only memory (CD-ROM), for example. The computer-readable media may also be any other volatile or non-volatile storage systems. A computer-readable medium may be considered a computer-readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

1. A system for label-free, non-destructive detection of pluripotency of cell colonies within a cell sample, the system comprising:
at least one processor; and
a non-transitory computer-readable medium, having stored therein instructions executable by the at least one processor to cause the system to perform operations comprising:
obtaining a phase contrast image of a cell sample that contains stem cells;
applying the phase contrast image to a first machine learning model to determine a set of locations and extents of a plurality of cell nuclei within the phase contrast image;
based on the phase contrast image, determining a first location and extent of a first cell colony of a plurality of cell colonies within the phase contrast image;
based on the portion of the phase contrast image that is within the first location and extent of the first cell colony and those of the determined set of locations and extents of cell nuclei that are within the location and extent of the first cell colony, determining a first set of features for the first cell colony; and
applying the first set of features to a second machine learning model to predict a first level of pluripotency of the first cell colony.

2. The system of claim 1, wherein the stem cells of the cell sample comprise at least one of induced pluripotent stem cells, mesenchymal stem cells, or embryonic stem cells.

3. The system of claim 1, wherein the second machine learning model comprises an ensemble model.

4. The system of claim 1, wherein applying the phase contrast image to the first machine learning model to determine the set of locations and extents of cell nuclei within the phase contrast image comprises:
applying the phase contrast image to the first machine learning model to generate a predicted pattern of a nucleus-specific fluorophore within the cell sample; and
segmenting the predicted pattern to determine the set of locations and extents of cell nuclei within the phase contrast image.

5. The system of claim 1, wherein determining the first location and extent of the first cell colony within the phase contrast image comprises applying the phase contrast image to a third machine learning model to determine the first location and extent of the first cell colony within the phase contrast image.

6. The system of claim 5, wherein applying the phase contrast image to the third machine learning model to determine the first location and extent of the first cell colony within the phase contrast image comprises:
applying the phase contrast image to the third machine learning model to determine the location and extent of a contiguous cell mass within the phase contrast image; and
identifying respective locations and extents of a plurality of sub-regions of the determined location and extent of the contiguous cell mass, wherein the first location and extent of the first cell colony is the determined location and extent of one of the sub-regions of the contiguous cell mass.

7. The system of claim 6, wherein a determined second location and extent of a second cell colony of the plurality of cell colonies within the phase contrast image is the determined location and extent of another one of the sub-regions of the contiguous cell mass, and wherein the operations further comprise:
based on the portion of the phase contrast image that is within the second location and extent of the second cell colony and those of the determined set of locations and extents of cell nuclei that are within the second location and extent of the second cell colony, determining a second set of features for the second colony; and
applying the second set of features to the second machine learning model to predict a second level of pluripotency of the second cell colony.

8. The system of claim 7, further comprising:
providing, on a display, an indication of the phase contrast image, the locations and extents of the first and second cell colonies within the phase contrast image, and the first and second levels of pluripotency of the first and second cell colonies, respectively.

9. The system of claim 7, wherein the operations further comprise:
responsive to determining that the first level of pluripotency exceeds a threshold value and that the second level of pluripotency does not exceed the threshold value, operating a robotic cell picker to extract cells from the first cell colony.

10. The system of claim 1, wherein the first set of features comprises at least one of: an area of the first cell colony, a convex area of the first cell colony, an eccentricity of the first cell colony, an extent of the first cell colony, a mean, minimum, maximum, or percentile value of an intensity of the portion of the phase contrast image that is within the first location and extent of the first cell colony, a perimeter length of the first cell colony, a solidity of the first cell colony, a Hu moment of the portion of the phase contrast image that is within the first location and extent of the first cell colony, a weighted Hu moment of the portion of the phase contrast image that is within the first location and extent of the first cell colony, a Haralick texture metric of the portion of the phase contrast image that is within the first location and extent of the first cell colony, a number of the nuclei within the first cell colony, a mean area of the nuclei within the first cell colony, a standard deviation of the areas of the nuclei within the first cell colony, a solidity of the nuclei within the first cell colony, or a nucleus-to-cytoplasm ratio of the first cell colony.

11. The system of claim 1, wherein obtaining the phase contrast image of the cell sample comprises phase contrast imaging a live cell sample while the live cell sample is located within an incubator.

12. The system of claim 11, wherein the phase contrast image is a first phase contrast image taken at a first point in time, wherein the first level of pluripotency is a level of pluripotency of the first cell colony at the first point in time, and wherein the operations further comprise:
phase contrast imaging the cell sample at a second point in time while the cell sample is located within the incubator to obtain a second phase contrast image of the cell sample, wherein the second point in time is after the first point in time;
applying the second phase contrast image to the first machine learning model to determine a second set of locations and extents of cell nuclei within the second phase contrast image;
based on the second phase contrast image, determining a third location and extent of the first cell colony within the second phase contrast image;
based on the portion of the second phase contrast image that is within the first location and extent of the first cell colony and those of the determined second set of locations and extents of cell nuclei that are within the third location and extent of the first cell colony, determining a third set of features for the first cell colony; and
applying the third set of features to the second machine learning model to predict a third level of pluripotency of the first cell colony at the second point in time.

13. The system of claim 11, wherein the operations further comprise, based on the first level of pluripotency and prior to the second point in time, at least one of (i) adjusting a rate at which a substance is continuously provided to the cell sample or (ii) providing a substance to the cell sample.

14. The system of claim 11, wherein the operations further comprise:
responsive to determining that the first level of pluripotency exceeds a threshold value, operating a robotic cell picker to extract cells from the first cell colony.

15. (canceled)

16. The system of claim 1, wherein the operations further comprise:
while the cell sample is located within an incubator, obtaining an additional plurality of phase contrast images of the cell sample at a plurality of respective different points in time;
based on the additional plurality of phase contrast images, using the first machine learning model and the second machine learning model to determine, for the first cell colony, a plurality of respective levels of pluripotency of the first cell colony at the respective different points in time; and
storing the first level of pluripotency and the plurality of respective levels of pluripotency of the first colony in a quality control record for the cell sample.

17. The system of claim 1, wherein a change in the level of pluripotency of the cell colonies is monitored in real-time.

18. A non-transitory computer readable medium having stored thereon program instructions executable by at least one processor to cause the at least one processor to perform a method comprising:
obtaining a phase contrast image of a cell sample that contains stem cells;
applying the phase contrast image to a first machine learning model to determine a set of locations and extents of a plurality of cell nuclei within the phase contrast image;
based on the phase contrast image, determining a first location and extent of a first cell colony of a plurality of cell colonies within the phase contrast image;
based on the portion of the phase contrast image that is within the first location and extent of the first cell colony and those of the determined set of locations and extents of cell nuclei that are within the location and extent of the first cell colony, determining a first set of features for the first cell colony; and
applying the first set of features to a second machine learning model to predict a first level of pluripotency of the first cell colony.

19. (canceled)

20. A system for non-destructively detecting pluripotency of a plurality of cell colonies within a live cell sample comprising:
at least one processor;
an incubator;
a phase contrast imager; and
a non-transitory computer-readable medium, having stored therein instructions executable by the at least one processor to cause the system to perform a method comprising:
operating the phase contrast imager to obtain a phase contrast image of the live cell sample that contains stem cells and that is located in the incubator;
applying the phase contrast image to a first machine learning model to generate a mask indicative of the presence of cell nuclei within the phase contrast image;
determining, based on the mask, locations and extents of cell nuclei within the phase contrast image;
applying the phase contrast image to a second machine learning model to determine locations and extents of the plurality of cell colonies within the phase contrast image;
based on the portion of the phase contrast image that is within the first location and extent of a first cell colony and those of the determined set of locations and extents of cell nuclei that are within the location and extent of the first cell colony; and
determining a plurality of respective levels of pluripotency for the plurality of cell colonies, wherein determining a level of pluripotency of a particular cell colony of the plurality of cell colonies comprises:
determining a set of features for the particular cell colony based on the portion of the phase contrast image that is within the determined location and extent of the particular cell colony and those of the determined locations and extents of cell nuclei that are within the location and extent of the particular cell colony, and
applying the set of features to a third machine learning model to predict a level of pluripotency of the particular cell colony.

21. The non-transitory computer readable medium of claim 18, wherein applying the phase contrast image to the first machine learning model to determine the set of locations and extents of cell nuclei within the phase contrast image comprises:
applying the phase contrast image to the first machine learning model to generate a predicted pattern of a nucleus-specific fluorophore within the cell sample; and
segmenting the predicted pattern to determine the set of locations and extents of cell nuclei within the phase contrast image.

22. The non-transitory computer readable medium of claim 18, wherein determining the first location and extent of the first cell colony within the phase contrast image comprises:
applying the phase contrast image to a third machine learning model to determine the location and extent of a contiguous cell mass within the phase contrast image; and
identifying respective locations and extents of a plurality of sub-regions of the determined location and extent of the contiguous cell mass, wherein the first location and extent of the first cell colony is the determined location and extent of one of the sub-regions of the contiguous cell mass.
